(19) United States
(12) Patent Application Publication
(10) Pub. No.: US 2006/0280281 A1
(43) Pub. Date: Dec. 14, 2006

(54) Method for calculating absorber-specific weighting coefficients and method for improving a contrast-to-noise ratio, dependent on an absorber, in an x-ray image, produced by an x-ray machine, of an object to be examined

(76) Inventors: Thomas Flohr, Uehlfeld (DE); Michael Grasruck, Erlangen (DE); Karl Stierstorfer, Erlangen (DE)

Correspondence Address: HARNESS, DICKEY & PIERCE, P.L.C., P.O.BOX 8910, RESTON, VA, 20195, US

(21) Appl. No.: 11/451,581

(22) Filed: Jun. 13, 2006

(30) Foreign Application Priority Data
Jun. 14, 2005 (DE) 10 2005 027 436.6

(51) Int. Cl. H05G 1/60; A61B 6/00; G01N 23/00; G21K 1/12

(52) U.S. Cl. 378/5

(57) ABSTRACT

A method is disclosed for calculating absorber-specific weighting coefficients and a method is disclosed for improving a contrast-to-noise ratio, dependent on an absorber, in an x-ray image of an object to be examined produced by an x-ray machine. A weighted summation of detector output signals from different energy windows of an energy-selector detector are used to improve the contrast-to-noise ratio as a function of the absorber.

### PRIORITY STATEMENT

The present application hereby claims priority under 35 U.S.C. §119 on German patent application number DE 10 2005 027 436.6 filed Jun. 14, 2005, the entire contents of which is hereby incorporated herein by reference.

### FIELD

The invention generally relates to a method for calculating absorber-specific weighting coefficients and/or to a method for improving a contrast-to-noise ratio, dependent on an absorber, in an x-ray image, produced by an x-ray machine, of an object to be examined.

### BACKGROUND

The contrast between various absorbers or substances of an object in an x-ray image produced by the x-ray machine is caused by the fact that the substances have different absorption properties relative to x-radiation. In the case of a medical diagnosis, it is frequently necessary to image a single substance relevant to the diagnosis, for example bone tissue or a contrast medium, in the x-ray image with a particularly high contrast-to-noise ratio. The quality of the x-ray image produced, and the success of a diagnosis in this case thus depend substantially on the achievable contrast-to-noise ratio between, specifically, a relevant substance and all remaining substances present in the object.

In order to acquire projections of the object that constitute the basis for reconstructing an x-ray image, use is generally made of energy-weighted detectors in the case of which the detector output signals detected in relation to each projection are substantially proportional to the energy of the x-radiation converted in the detector. With such detectors, a contrast-to-noise ratio in the x-ray images that is dependent on the absorber can be adapted only by physical x-ray measures such as appropriate filtering, selection of a tube voltage or a tube current, or by selecting a suitable detector material.

US 2004/0101087 A1 discloses, for example, a tomography unit for detecting 3D structures with the aid of which the contrast-to-noise ratio between different absorbers is improved in the reconstructed x-ray image by virtue of the fact that for each projection direction two projections are detected separately from one another in relation to differently set tube voltages, and subtracted.

### SUMMARY

It is an object of at least one embodiment of the present invention to specify a method for an x-ray machine with the aid of which the possibility is provided of improving a contrast-to-noise ratio in an x-ray image produced by an x-ray machine; for example doing so as a function of an absorber and with simple devices/methods.

An object may be achieved by a method for calculating absorber-specific weighting coefficients for improving a contrast-to-noise ratio dependent on an absorber.

Moreover, an object may be achieved by a method for improving a contrast-to-noise ratio dependent on an absorber.

The inventors have realized, in at least one embodiment, that the achievable contrast-to-noise ratio can be improved as a function of an absorber, in an x-ray image, produced by an x-ray machine by weighting an x-radiation passing through the object as function of an energy range. Owing to the different weighting of the energy ranges of the x-radiation, it is possible, in particular, to weight more strongly those ranges that make a stronger contribution to the contrast of a relevant absorber, for example, bone tissue or iodine, to the remaining absorbers in the object, for example surrounding soft part tissue.

X-radiation in different energy ranges can in this case be detected by way of an energy-resolving detector that has a plurality of energy windows. Suitable weighting coefficients can be derived in this case from two spectra of the x-radiation on the basis of detector output signals of the energy-resolving detector, the first spectrum being obtained by way of an object with the relevant absorber, and the second spectrum being obtained by way of an object without this relevant absorber.

According to at least one embodiment of the invention, the method for calculating absorber-specific weighting coefficients for improving the contrast-to-noise ratio, dependent on an absorber, in the x-ray image, produced by the x-ray machine, of the object to be examined, the x-ray machine including an energy-resolving detector with a plurality of detector elements, which has at least two energy windows in which different energy ranges of the x-radiation passing through the object are detected, the method comprising steps in which
a) the first spectrum is determined for a first reference object without the absorber, a detector output signal assigned to the first spectrum being determined in relation to each of the two energy windows of the detector,
b) the second spectrum being determined for a second reference object with the absorber, a detector output signal assigned to the first spectrum being determined in relation to each of the two energy windows of the detector, and in which
c) the absorber-specific weighting coefficient corresponding to the energy window of the detector is respectively calculated in relation to each energy window of the detector from the determined detector output signals of the first and second spectrum.

The absorber-specific weighting coefficients can therefore be provided in a simple way for different absorbers with simple devices/methods for improving the contrast-to-noise ratio in the x-ray image.

The weighting coefficients can optionally be determined either experimentally from produced spectra to both reference objects without a large numerical outlay, or by way of simulation. In both cases, the calculation of the weighting coefficients takes place on the basis of the detector output signals, determined for the two spectra, in relation to the different energy windows of the detector.

In the case of simulation, the first step is to use a numerical model to determine the x-radiation spectrum produced by an x-ray source, then the x-radiation spectrum after passage through the reference object is calculated by taking account of the absorption properties, and subsequently the detector output signals in the different energy windows are simulated in relation to the x-radiation spectrum thus calculated by taking account of the corresponding response functions of the detector.

A contrast-to-noise ratio dependent on the absorber can be improved with high flexibility with reference to a contrast relevant to the diagnosis by the provision of absorber-specific weighting functions.

In addition to high flexibility with reference to a specific medical problem in which it is necessary to visualize a specific absorber, for example bone tissue or contrast medium, in an x-ray image, the provision of the absorber-specific weighting coefficients yields a prescribed contrast-to-noise ratio by comparison with a conventionally obtained x-ray image in conjunction with a lesser x-ray dose such that the object, for example a patient, is exposed to a lesser radiation burden during diagnosis.

The absorber-specific weighting coefficients may be calculated, for example, using the following computing rule:
wk=(n1k−n2k)/(n1k+n2k),
k being an index for distinguishing the energy windows, wk representing the absorber-specific weighting coefficient of the energy window k, and n1k specifying the detector output signal of the first spectrum for the energy window k, and n2k specifying the detector output signal of the second spectrum for the energy window k.

Such a computing rule ensures that a weighting coefficient is larger the larger the difference in the spectra between the two reference objects in the corresponding energy window of the detector, or the larger the contribution of the energy range of the x-radiation to the contrast between the absorber relevant to the examination and the remaining absorbers.

In an advantageous variant of at least one embodiment of the invention, the absorber-specific weighting coefficients are loaded from a database such that the contrast-to-noise ratio can be dynamically adapted to any desired absorber as a function of the medical problem. Thus, for example, it would be conceivable to use one and the same detector output signals to produce in sequence x-ray images in which the contrast is improved for different absorbers. In order to examine bone structures, the absorber may, for example, exhibit an attenuation property of bone. In a further advantageous variant of at least one embodiment of the invention, the absorber can, however, also exhibit the attenuation property of iodine by dynamically switching over the absorber-specific weighting coefficients such that the distribution of a contrast medium in the interior of the body can be analyzed.

Detector output signals can be simultaneously detected in a number of energy windows in a simple way by way of a counting semiconductor detector.

According to at least one embodiment of the invention, the calculated absorber-specific weighting coefficients can be used for a method for improving a contrast-to-noise ratio, dependent on the absorber, in an x-ray image, produced by an x-ray machine, of the object to be examined, the x-ray machine comprising the energy resolving detector with a plurality of detector elements, which has at least two energy windows in which different energy ranges of an x-radiation passing through the object are detected, in which
a) a detector output signal is respectively detected in relation to each detector element for the at least two different energy windows of the detector as a measure of the intensity of the x-radiation in the corresponding energy range,
b) the detector output signals, assigned to the respective detector element of the two different energy windows are weighted with absorber-specific weighting coefficients and summed up such that a corrected detector output signal results in relation to each detector element, and in which
c) the corrected detector output signals are calculated to form an x-ray image in which a contrast-to-noise ratio dependent on the absorber is improved.

As already mentioned, a contrast-to-noise ratio dependent on the absorber can be improved with high flexibility with reference to a contrast relevant to the diagnosis by a simple weighting of the detected detector output signals of the energy resolving detector.

In addition to high flexibility with reference to a specific medical problem in which it is necessary to visualize a specific absorber in an x-ray image, as already mentioned a prescribed contrast-to-noise ratio by comparison with a conventionally obtained x-ray image is achieved in conjunction with a lesser x-ray dose such that the object, for example a patient, is exposed to a lesser radiation burden.

### BRIEF DESCRIPTION OF THE DRAWINGS

Example embodiments of the invention and further advantageous refinements of the invention in accordance with the subclaims are illustrated in the following schematics. In the drawings:

FIG. 1 shows in a perspective view an x-ray machine that is suitable for carrying out the method according to at least one embodiment of the invention for calculating absorber-specific weighting coefficients and for improving the contrast-to-noise ratio in an x-ray image,

FIG. 2 shows two spectra, used for calculating the absorber-specific weighting coefficients, of a first reference object without an absorber, and of a second reference object with an absorber in the form of iodine,

FIG. 3 shows response functions of various energy windows of a quantum-counting detector as a function of a quantum energy of an x-radiation, in the form of a sketch,

FIG. 4 shows the first and the second spectrum of the first and second reference object together with the absorber-specific weighting coefficients determined in relation to the various energy windows, in a diagram,

FIG. 5 shows a comparison of the signal response of the detector for the two spectra of the reference object before and after weighting,

FIG. 6 shows a flowchart of the method according to at least one embodiment of the invention for calculating absorber-specific weighting coefficients, in the form of a sketch, and

FIG. 7 shows a flowchart of the method according to at least one embodiment of the invention for improving a contrast-to-noise ratio, in the form of a sketch.

### DETAILED DESCRIPTION OF THE EXAMPLE EMBODIMENTS

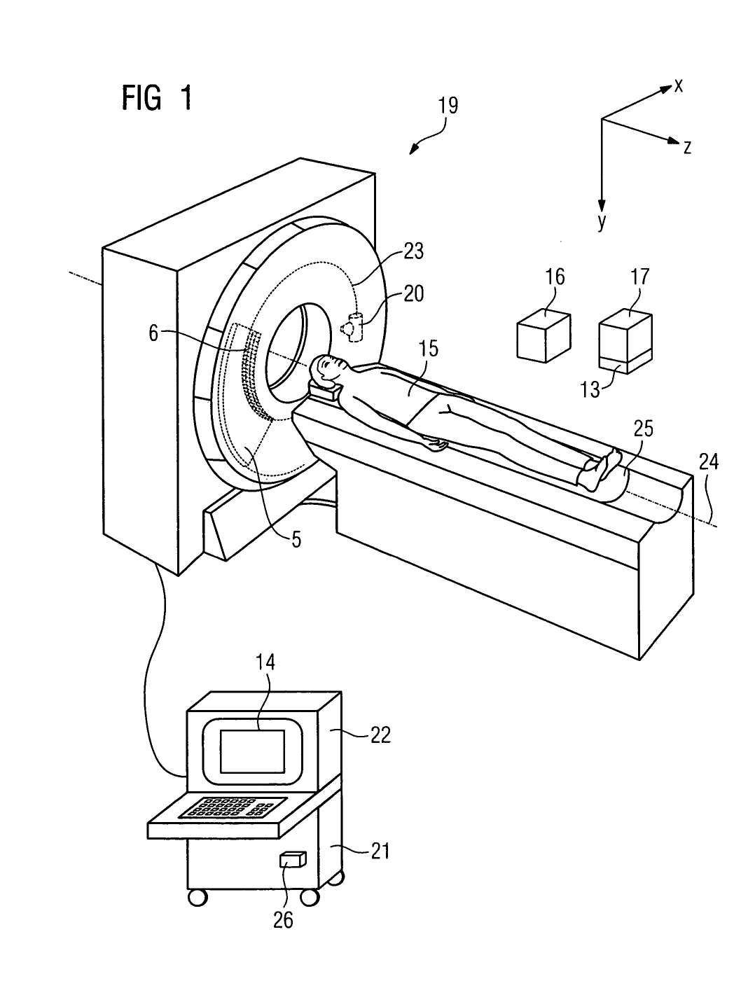

In the form, here, of a computed tomography unit 19, FIG. 1 shows in a perspective view an x-ray machine that is suitable for executing the method according to at least one embodiment of the invention for calculating absorber-specific weighting coefficients 1, 2, 3, 4 and for improving the contrast-to-noise ratio in an x-ray image 14. The computed tomography unit 19 essentially includes an x-ray source 20 in the form of an x-ray tube 1, an energy resolving detector 5 that has detector elements 6 arranged in a detector array as columns and as rows, only one thereof being provided with a reference numeral, a computing device 21 for calculating the absorption specific weighting coefficients 1, 2, 3, 4 and for improving the contrast-to-noise ratio, and a display unit 22 for displaying the x-ray image 14 produced. The x-radiation produced by the x-ray source 20 in the form of an x-ray tube is set by means of a prescribable input value in the form of a tube current.

The x-ray tube 10 and the detector 5 are part of a recording system and are fitted on a rotary frame 23 in a fashion lying opposite one another in such a way that during operation of the computed tomography unit 19 an x-ray beam emanating from a focus of the x-ray tube 20 and delimited by marginal rays impinges on the detector 5.

The rotary frame 23 can be set rotating about a rotation axis 24 by way of a drive device (not illustrated). Here, the rotation axis 24 runs parallel to the z-axis of a spatial rectangular coordinate system illustrated in FIG. 1. It is possible in this way to prepare projections from different projection directions or rotary angle positions of the recording system in order to reconstruct a volume image for an object 15, for example a patient, located on a measuring table 25.

By way of a tube current set by the arithmetic lodge unit 21 and converted by a generator, the x-ray tube 20 produces a spectrum, characteristic of the x-ray tube, of the x-radiation that transirradiates an object 15 positioned in the measurement area, and is partially absorbed by said object, and which subsequently strikes the detector element 6 of the energy-selector detector 5.

The absorber-specific weighting coefficients 1, 2, 3, 4 can be loaded dynamically from a database 26 such that a contrast-to-noise ratio can be specifically improved for a particular absorber 13 as a function of the examination to be carried out. Moreover, FIG. 1 also illustrates by way of example two different reference objects 16, 17 by way of which the absorber-specific weighting coefficients 1, 2, 3, 4 can be determined.

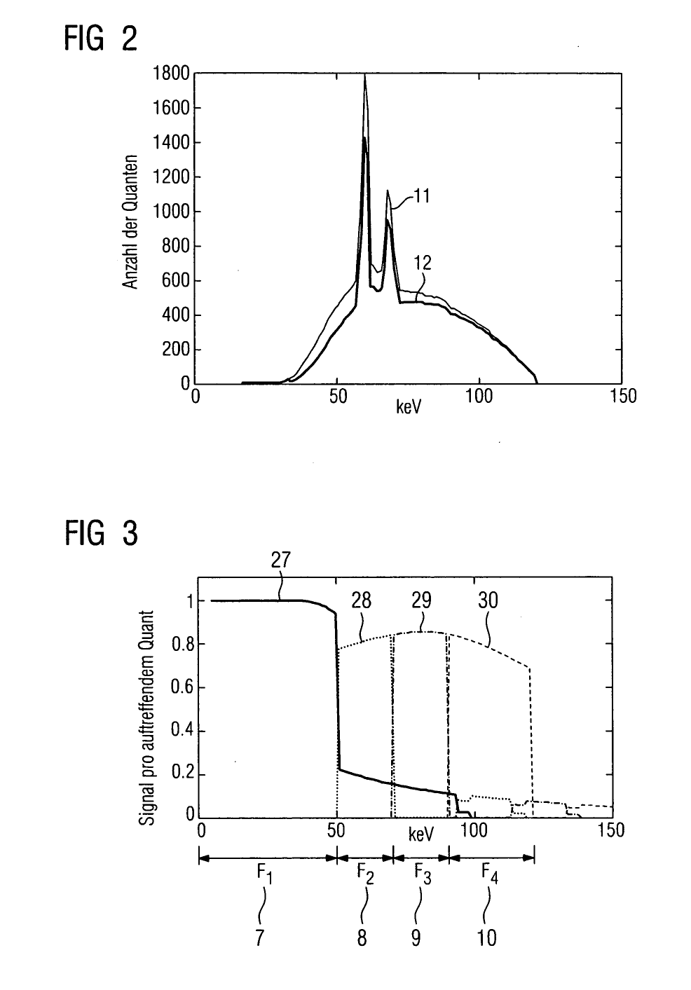

By way of example, FIG. 2 shows two spectra 11, 12 of an x-radiation, passing through an object 15 and impinging on the detector 5, for the two different reference objects 16, 17, that are used to calculate the absorber-specific weighting coefficients 1, 2, 3, 4 in the case of a set tube voltage of 120 kV, the energy of the x-radiation being plotted along the x axis in units of keV, and the intensity of the x-radiation being plotted as the number of incoming x-ray quanta along the y-axis.

The thin line marks the spectrum assigned to the first reference object 16, which exhibits the general absorption properties of the object 15 to be examined. In this example, the absorption properties of the object 15 to be examined are simulated by a layer of water 200 mm thick, and a layer of aluminum 3 mm thick. The thick line in FIG. 2, in contrast, marks the spectrum assigned to the second reference object 17, which, in addition to the general absorption properties of the object 15, exhibits the absorption property of the relevant absorber 13 that is to be imaged in the x-ray image 14 with a higher contrast-to-noise ratio.

The aim in this example embodiment is, by way of example, to produce a particularly good contrast between an absorber 13 in the form of iodine and the object 15 in the x-ray image 14 in order to examine a distribution of a contrast medium in the object 15. For this reason, the second reference object 17 contains 0.03 g/cm³ of iodine in addition to the substances of the first reference body. Iodine is merely of an example nature in the context of the example embodiment. Absorption specific weighting coefficients 1, 2, 3, 4 for improving a contrast-to-noise ratio can fundamentally be determined for any other desired substances.

In principle, the visible contrast in an x-ray image 14 between the absorber 13 and the object 15 is larger the larger the difference in the intensity of the x-radiation. As may be gathered from FIG. 1, the difference in the intensity of the x-radiation between the two spectra 11, 12 of the reference objects 16, 17 is a function of the energy of the x-radiation. Above an energy of approximately 100 keV for the x-radiation, the two spectra 11, 12 become evermore identical, while a substantial difference in the x-radiation can be observed in an energy interval between 40 keV and 60 keV.

The inventors realized that given an appropriate weighting of detector output signals that represent the intensity of the x-radiation in different energy ranges, it is possible to improve the contrast-to-noise ratio in an x-ray image 14 by taking greater account of energy ranges of the x-radiation with a high difference between the spectrum of the object and the spectrum of the absorber, than of energy ranges with only a slight difference.

Detector output signals relating to different energy ranges of the x-radiation can, for example, be detected by way of an energy selector detector 5 that has a plurality of energy windows 7, 8, 9, 10.

The detector 5 used in this example embodiment is a semiconductor detector with four different energy windows 7, 8, 9, 10 in which the intensity of the x-radiation of a specific energy range is respectively detected. The four energy windows 7, 8, 9, 10 of the semiconductor detector, based on gadolinium, for example, can be formed by four sequentially arranged detector planes, an absorption filter in the form of a copper filter being arranged in each case between the planes for the purpose of reducing the energy of the x-radiation. It is possible in this way to produce for each detector element four detector output signals that represent the intensity of the x-radiation for different energy ranges. However, it would likewise be conceivable to use a semiconductor detector that records each individual event on the basis of a very high time resolution such that the energy of each incoming x-ray quantum can be determined.

Shown in FIG. 3 as a function of a quantum energy of the x-radiation are the response functions 27, 28, 29, 30 of a quantum-counting semiconductor detector that has a total of four energy windows 7, 8, 9, 10, the quantum energy of x-radiation being plotted in units of keV along the x-axis, and the signal per impinging quantum of x-radiation being plotted along the y-axis. The energy thresholds for which substantially no signal is produced in relation to an energy window lie at 50, 70, 90 and 120 keV, but can differ substantially from these values as a function of the detector 5 used. It is a striking fact that the response functions 27, 28, 29, 30 of the individual energy windows 7, 8, 9, 10 above the threshold energy do not drop completely to zero. The reason for this can be that the energy converted in the detector 5 can drop below the corresponding energy threshold of an energy window 7; 8; 9; 10 because of interactions between the x-ray quanta and the atoms of the semiconductor material of the detector 5. However, this state of affairs, which is also denoted as K escape, plays a very subordinate role in the method according to at least one embodiment of the invention and need not be considered further.

Thus, in this example embodiment four detector output signals are detected in relation to each detector elements 6 and to a prescribed spectrum 11; 12 of the x-radiation that represent the intensity of the x-rays in different, substantially juxtaposed energy ranges. In order to improve the contrast-to-noise ratio, achievable in an x-ray image 14, for a specific absorber 13, it is necessary to determine suitable absorber-specific weighting coefficients 1, 2, 3, 4 with the aid of which the detector output signals are weighted and subsequently summed up.

A mathematical relationship is specified below with the aid of which suitable absorber-specific weighting coefficients 1, 2, 3, 4 can be determined on the basis of the first spectrum 11 of the first reference object. 16 without the absorber, and of the second spectrum 12 with the absorber 17, by taking account of the response functions 27, 28, 29, 30 of the detector 5.

The detector output signal n1k for the energy window k with the response function Dk in relation to the spectrum Si of the x-radiation is calculated according to the following equation:
nᵢₖ=∫Sᵢ(E)Dₖ(E)dE, (1)
nik being the detector output signal, Si being the spectrum of the ith reference object, Dk being the response function of the kth energy window, and E being the energy of the x-radiation.

A corrected detector output signal Ni is yielded in very general terms from a weighting, still to be determined, of the detector output signals of a detector element:

$${N}_{i} = {\sum }_{k} {w}_{k} \cdot {n}_{ik} , \qquad ( 2 )$$

Ni being the corrected detector output signal of the ith reference object, wk being the absorber-specific weighting coefficient, yet to be determined, of the energy window k, and nik being the detector output signal of the ith reference object in relation to the energy window K.

In the case of a quantum-counting detector, the noise can be calculated from the roots of the detected quanta in accordance with the following equation:
σᵢₖ²=nᵢₖ, (3)
sik being the noise of the detector output signal, and nik being the detector output signal of the ith spectrum in relation to the energy window k.

It is thereby possible to specify the following contrast-to-noise ratio for the two corrected signals in relation to the two spectra of the reference object:

$${CNR}^{2} = \frac{{[ {N}_{1} - {N}_{2} ]}^{2}}{{σ}_{{N}_{1}}^{2} + {σ}_{{N}_{2}}^{2}} = \frac{{[ {\sum }_{k} {w}_{k} \cdot ( {n}_{1 k} - {n}_{2 k} ) ]}^{2}}{{\sum }_{k} {w}_{k}^{2} \cdot ( {n}_{1 k} + {n}_{2 k} )} , \qquad ( 4 )$$

CNR being the contrast-to-noise ratio of a specific absorber, which is to be maximized, N1 and N2 respectively being the corrected detector output signal in relation to the first and second reference object, s1k and s2k respectively being the noise of the detector output signal in relation to the first and second reference object for the energy window k, n1k and n2k respectively being the detector output signal of the first and second spectrum in relation to the energy window k, and wk being the absorber-specific weighting coefficient being sought in relation to the energy window k.

The denominator of equation (4) is calculated here from the Gaussian error propagation formula by using equations (2) and (3).

The absorber-specific weight coefficients suitable for improving the contrast-to-noise ratio can be determined using an optimization method known per se, for example on the basis of a first partial derivative with respect to the weighting coefficients being sought, and lead to the following result:

$${w}_{k} = \frac{{n}_{1 k} - {n}_{2 k}}{{n}_{1 k} + {n}_{2 k}} . \qquad ( 5 )$$

The absorber-specific weighting coefficients 1, 2, 3, 4 can thus be calculated in a simple manner separately for each energy window 7; 8; 9; 10, without a large numerical outlay, from the detector output signals that have been determined in relation to the two reference objects 16, 17 with and without the absorber 13. It is of no importance here whether the detector output signals have been obtained experimentally by irradiating appropriately prepared reference objects 16, 17, or by way of simulation.

Calculating the absorber-specific weighting coefficients 1, 2, 3, 4 by using equation (5) leads to the following result for the example embodiment described here:
w1=0.45, w2=0.31, w3=0.16 and w4=0.08.

The contrast-to-noise ratio can therefore be substantially improved by a weighted summation of the detector output signals per detector element. A contrast-to-noise ratio is achieved in this case that has improved by 24% by comparison with an x-ray image 14 that has been determined on the basis of constant weighting coefficients, and this would permit a reduction of 24% in dosage.

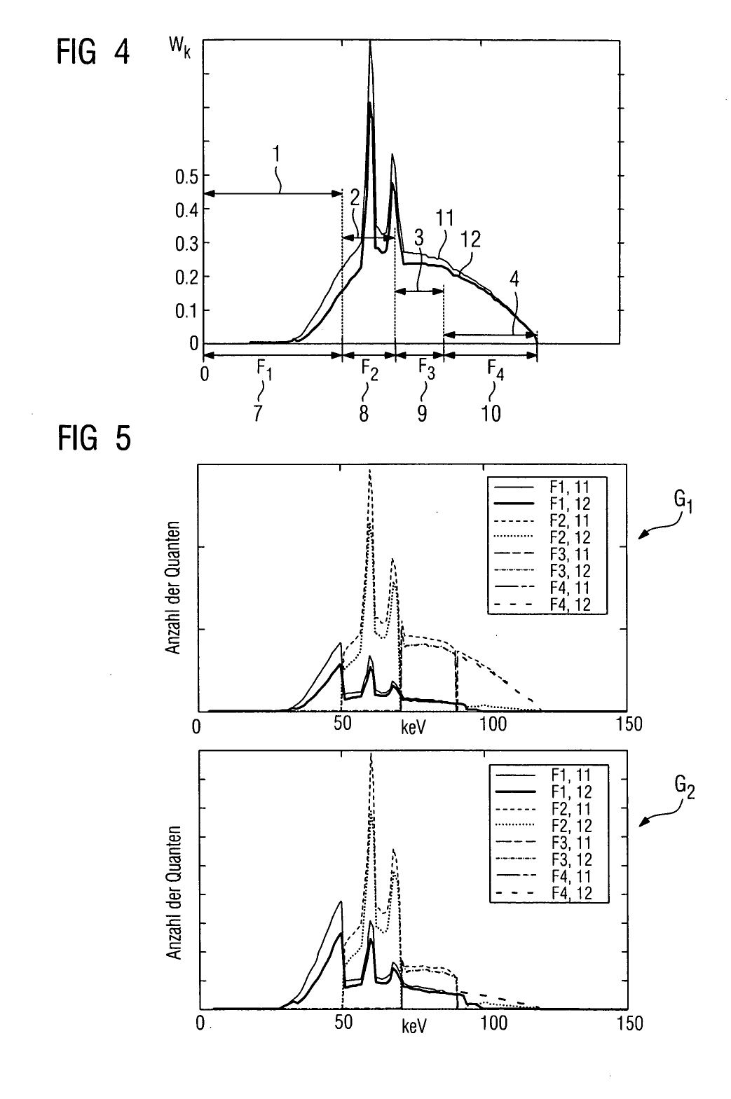

Plotted in a diagram in FIG. 4 are the determined absorber-specific weighting coefficients 1, 2, 3, 4 of the various energy windows 7, 8, 9, 10 of the detector 5 together with the two spectra 11, 12 of the reference objects 16, 17, the different energy windows 7, 8, 9, 10 being plotted in the x-direction, and the magnitude of the weighting coefficient 1, 2, 3, 4 being plotted in the y-direction. As is to be gathered from the diagram, the absorber-specific weighting coefficient 1; 2; 3; 4 for an energy window 7; 8; 9; 10 of the detector 5 is larger the larger the difference between the two spectra 11, 12 in the energy window 7; 8; 9; 10 or the larger the contribution of the corresponding energy window 7; 8; 9; 10 to the contrast-to-noise ratio dependent on the absorber 13.

FIG. 5 shows by way of example the effect of a weighting of the signal response of the detector performed using the procedure last described. The coordinate axes were adopted in a way corresponding to FIG. 2. The differently marked line segments respectively represent a signal response of the detector in relation to a specific energy window 7; 8; 9; 10 as a function of the respective spectrum 11; 12. As is to be seen from the two graphs G1 and G2, the weighting of the signal response in the different energy windows 7; 8; 9; 10 of the detector 5 with the absorber-specific weighting coefficients 1, 2, 3, 4 evaluates more strongly those energy ranges that make a stronger contribution to the contrast-to-noise ratio dependent on the absorber 13. Specifically, a higher contribution to the contrast-to-noise ratio of an energy range is obtained whenever the difference between the signal responses between the two spectra 11, 12 is particularly high for an energy range.

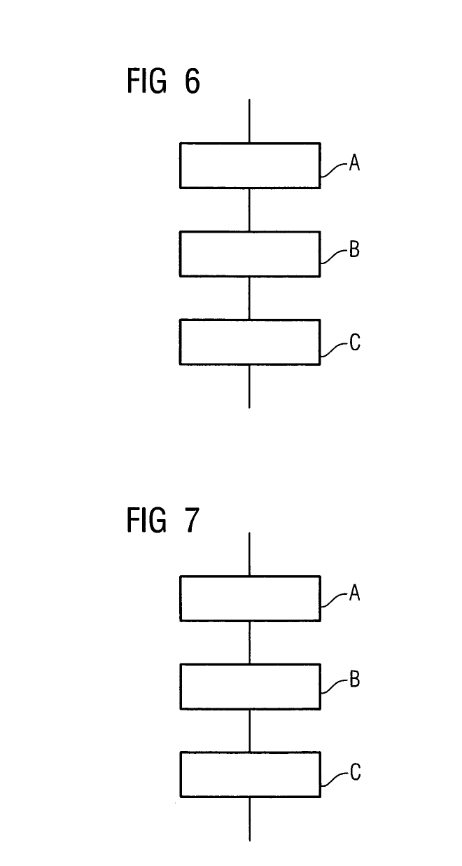

The method for calculating the absorber-specific weighting coefficient 1, 2, 3, 4 is represented in FIG. 6 in summary fashion in relation to what has just been said in the form of a block diagram for the case in which the energy selective detector 5 has two energy windows:

In the method, in a first step A a first spectrum is determined for a first reference object without the absorber, and a detector output signal assigned to the first spectrum is determined in relation to each of the two energy windows of the detector,

In the method, in a method step B, a second spectrum is determined for a second reference object with the absorber, and a detector output signal assigned to the first spectrum is determined in relation to each of the two energy windows of the detector, and

the absorber-specific weighting coefficient corresponding to the energy window of the detector is calculated in a subsequent method step C in relation to each energy window of the detector from the determined detector output signals of the first and the second spectrum.

Absorber-specific weighting coefficients can be determined for a plurality of different substances and be stored in a database 26 assigned to the x-ray machine, and can be read out dynamically if required from the memory in order to calculate an x-ray image in which the contrast-to-noise ratio relating to a corresponding absorber is to be improved.

The method of at least one embodiment, for improving the contrast-to-noise ratio in an x-ray image is illustrated in the form of block diagram in FIG. 7 for the case in which the detector has two energy windows. The method includes a step A in which, in relation to each detector element, a detector output signal is detected for the at least two different energy windows of the detector as a measure of the intensity of the x-radiation in the corresponding energy region, a method step B in which the detector output signals, assigned to the respective detector element, of the two different energy windows are weighted with the aid of absorber-specific weighting coefficients and summed up such that a correct detector output signal is yielded in relation to each detector element, and a final method step C in which the corrected detector output signals are calculated to produce an x-ray image in which a contrast-to-noise ratio dependent on the absorber is improved.

The basic idea of at least one embodiment of the invention can be summarized as follows:

At least one embodiment of the invention relates to a method for calculating absorber-specific weighting coefficients 1, 2, 3, 4 and to a method for improving a contrast-to-noise ratio, dependent on an absorber 13, in an x-ray image 14, produced by an x-ray machine, of an object 15 to be examined, the possibility being provided of using a weighted summation of detector output signals from different energy windows 7, 8, 9, 10 of an energy selector detector 5 to improve the contrast-to-noise ratio with the aid of simple devices/methods as a function of the absorber 13.

Further, elements and/or features of different example embodiments may be combined with each other and/or substituted for each other within the scope of this disclosure and appended claims.

Further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for calculating absorber-specific weighting coefficients for improving a contrast-to-noise ratio, dependent on an absorber, in an x-ray image of an object to be examined produced by an x-ray machine, the x-ray machine including an energy-selector detector with a plurality of detector elements and at least two energy windows in which different energy ranges of an x-radiation passing through the object are detectable, the method comprising:
determining a first spectrum for a first reference object without the absorber, a detector output signal assigned to the first spectrum being determined in relation to each of the at least two energy windows of the detector;
determining a second spectrum for a second reference object with the absorber, a detector output signal assigned to the second spectrum being determined in relation to each of at least two the two energy windows of the detector; and
respectively calculating the absorber-specific weighting coefficient corresponding to an energy window of the detector, in relation to each other energy window of the detector, from the determined detector output signals of the first and second spectrum.

2. The method as claimed in claim 1, wherein the absorber-specific weighting coefficient is calculated as follows:
wk=(n1k−n2k)/(n1k+n2k),
k being an index for distinguishing the energy windows, wk representing the absorber-specific weighting coefficient of the energy window k, and n1k specifying the detector output signal of the first spectrum for the energy window k, and n2k specifying the detector output signal of the second spectrum for the energy window k.

3. The method as claimed in claim 1, wherein the absorber-specific weighting coefficients are loaded from a database.

4. The method as claimed in claim 1, wherein the absorber used exhibits an attenuation property of bone.

5. The method as claimed in claim 1, wherein the absorber used exhibits an attenuation property of iodine.

6. The method as claimed in claim 1, wherein the energy-selector detector used to detect the detector output signals is a counting semiconductor detector.

7. The method as claimed in claim 1, wherein the x-ray machine used is a computed tomography unit.

8. A method for improving a contrast-to-noise ratio, dependent on an absorber, in a formed x-ray image of an object to be examined produced by an x-ray machine, the x-ray machine including an energy-selector detector with a plurality of detector elements and at least two energy windows in which different energy ranges of an x-radiation passing through the object are detected, the method comprising:
respectively detecting a detector output signal for the at least two different energy windows of the detector, in relation to each detector element, as a measure of an intensity of x-radiation in the corresponding energy range;
weighting the detector output signals, assigned to the respective detector element of the at least two different energy windows, with absorber-specific weighting coefficients and summing up the weighted detector output signals to produce a corrected detector output signal for each detector element; and
forming, from the corrected detector output signals, an x-ray image.

9. The method as claimed in claim 8, wherein the absorber-specific weighting coefficients are calculated by:
determining a first spectrum for a first reference object without the absorber, a detector output signal assigned to the first spectrum being determined in relation to each of the at least two energy windows of the detector;
determining a second spectrum for a second reference object with the absorber, a detector output signal assigned to the second spectrum being determined in relation to each of at least two the two energy windows of the detector; and
respectively calculating the absorber-specific weighting coefficient corresponding to an energy window of the detector, in relation to each other energy window of the detector, from the determined detector output signals of the first and second spectrum.

10. The method as claimed in claim 8, wherein the absorber-specific weighting coefficients are loaded from a database.

11. The method as claimed in claim 2, wherein the absorber used exhibits an attenuation property of bone.

12. The method as claimed in claim 2, wherein the absorber used exhibits an attenuation property of iodine.

13. The method as claimed in claim 9, wherein the absorber-specific weighting coefficients are loaded from a database.

14. A computer program to, when executed on a computer, cause the computer to carry out the method as claimed in claim 1.

15. A computer program product, including the computer program of claim 14.

16. A computer readable medium including program segments for, when executed on a computer, causing the computer to implement the method of claim 1.

17. A computer program to, when executed on a computer, cause the computer to carry out the method as claimed in claim 8.

18. A computer program product, including the computer program of claim 17.

19. A computer readable medium including program segments for, when executed on a computer, causing the computer to implement the method of claim 8.

20. An x-ray machine comprising:
an energy-selector detector with a plurality of detector elements and at least two energy windows in which different energy ranges of an x-radiation passing through an object to be examined are detectable;
means for determining a detector output signal assigned to a first spectrum, for a first reference object without an absorber, in relation to each of the at least two energy windows of the detector;
means for determining a detector output signal assigned to a second spectrum, for a second reference object with the absorber, in relation to each of the at least two energy windows of the detector; and
means for respectively calculating an absorber-specific weighting coefficient corresponding to an energy window of the detector, in relation to each other energy window of the detector, from the determined detector output signals of the first and second spectrum.

21. The x-ray machine as claimed in claim 20, wherein the absorber-specific weighting coefficient is calculated as follows:
wk=(n1k−n2k)/(n1k+n2k),
k being an index for distinguishing the energy windows, wk representing the absorber-specific weighting coefficient of the energy window k, and n1k specifying the detector output signal of the first spectrum for the energy window k, and n2k specifying the detector output signal of the second spectrum for the energy window k.

22. An x-ray machine comprising:
an energy-selector detector with a plurality of detector elements and at least two energy windows in which different energy ranges of an x-radiation passing through an object to be examined are detectable;
means for respectively detecting a detector output signal for the at least two different energy windows of the detector, in relation to each detector element, as a measure of an intensity of x-radiation in the corresponding energy range;
means for weighting the detector output signals, assigned to the respective detector element of the at least two different energy windows, with absorber-specific weighting coefficients and summing up the weighted detector output signals to produce a corrected detector output signal for each detector element; and
means for forming, from the corrected detector output signals, an x-ray image.

23. The x-ray machine as claimed in claim 22, wherein the absorber-specific weighting coefficients are calculated by:
determining a first spectrum for a first reference object without the absorber, a detector output signal assigned to the first spectrum being determined in relation to each of the at least two energy windows of the detector;
determining a second spectrum for a second reference object with the absorber, a detector output signal assigned to the second spectrum being determined in relation to each of at least two the two energy windows of the detector; and
respectively calculating the absorber-specific weighting coefficient corresponding to an energy window of the detector, in relation to each other energy window of the detector, from the determined detector output signals of the first and second spectrum.

24. A method for calculating absorber-specific weighting coefficients for improving a contrast-to-noise ratio, dependent on an absorber, in an x-ray image of an object to be examined, produced by an x-ray machine including an energy-selector detector with a plurality of detector elements and at least two energy windows in which different energy ranges of an x-radiation passing through the object are detectable, the method comprising:
determining a detector output signal, assigned to a first spectrum for a first reference object without the absorber, in relation to each of the at least two energy windows of the detector;
determining a detector output signal, assigned to a second spectrum for a second reference object with the absorber, in relation to each of at least two the two energy windows of the detector; and
respectively calculating the absorber-specific weighting coefficient corresponding to an energy window of the detector, in relation to each other energy window of the detector, from the determined detector output signals of the first and second spectrum.

25. The method as claimed in claim 24, wherein the absorber-specific weighting coefficient is calculated as follows:
wk=(n1k−n2k)/(n1k+n2k),
k being an index for distinguishing the energy windows, wk representing the absorber-specific weighting coefficient of the energy window k, and n1k specifying the detector output signal of the first spectrum for the energy window k, and n2k specifying the detector output signal of the second spectrum for the energy window k.

26. A computer program to, when executed on a computer, cause the computer to carry out the method as claimed in claim 24.

27. A computer program product, including the computer program of claim 26.

28. A computer readable medium including program segments for, when executed on a computer, causing the computer to implement the method of claim 24.
